(19) United States
(12) Patent Application Publication
(10) Pub. No.: US 2007/0213587 A1
(43) Pub. Date: Sep. 13, 2007

(54) Endo-Loop For Endoscope Surgery

(76) Inventor: Hwa-sook Moon, Busan (KR)

Correspondence Address: John W Renner;Renner Otto Boisselle & Sklar, 19th Floor, 1621 Euclid Avenue, Cleveland, OH, 44115, US

(21) Appl. No.: 11/587,606

(22) PCT Filed: Apr. 26, 2005
(86) PCT No.: PCT/KR05/01192

§ 371 Date: Oct. 27, 2006

(30) Foreign Application Priority Data
Apr. 30, 2004 (KR) 10-2004-0030678

(51) Int. Cl. A61B 17/04

(52) U.S. Cl. 600/138

(57) ABSTRACT

Disclosed is an endo-loop for an endoscopic surgery. The endo-loop includes: an insert rod inserted into a human body, the insert rod having a shape of a long, thin, and hollow bar; a suturing thread inserted through an interior of the insert rod; a suturing means provided at a tip end of the insert rod, the suturing means including at least one of the suturing thread, a suturing loop, a suturing needle connected to the suturing thread, and their combination; a suturing thread fixing member for fixing the suturing thread to the insert rod, the suturing thread extending backward along the insert rod, the suturing thread fixing member being detachably engaged with an open rear end portion of the inserted rod; and a spool on which the suturing thread extending outward through the open rear end portion of the insert rod is wound.

### TECHNICAL FIELD

The present invention relates to an endo-loop inserted into the interior of a human body together with an endoscope during an endoscopic surgery to suture a surgical portion, and more particularly to an endo-loop which can remarkably reduce the amount of the disused surgery materials by minimizing the number of endo-loops used in an endoscopic surgery, can efficiently perform the endoscopic surgery by preventing problems due to exchanges of endo-loops in different surgeries and confusion in the surgery, can reduce the manufacturing cost of the endo-loop and maximize functionality and feasibility of the endo-loop by manufacturing almost all kinds of endo-loops in one packing unit, by extending outward a suturing thread to the rear side of an insert rod having a suturing means at the tip end of the insert rod, detachably engaging a suturing thread fixing member with the insert rod, and winding the suturing thread extending outward by a predetermined distance on a spool.

### BACKGROUND ART

In general, endoscope is the general term for medical instruments which enables a person to see inner surfaces of hollow internal organs such as stomachs or gullets and interiors of portions of a body such as thoracic cavities or abdominal cavities. The endoscope is sorted into a bronchoscope, a laryngoscope, an esophagoscope, a stomach camera, a hysteroscope, a urethroscope, an arthroendoscope, a cystoscope, a proctorscope, a celioscope, a cardioscope, etc. according to their uses. The endoscope is a medical auxiliary instrument which helps discovery, diagnosis, and medical treatment of an illness by observing states of interiors of a body and facilitating resection of tissues and extraction of secretions.

In case that an internal organ, a blood vessel, or a nerve system is incised or sutured for the purpose of treating an illness generated in a thoracic cavity, an abdominal cavity, or a predetermined portion of a human body by using the endoscope, in the state in which an auxiliary instrument for surgery called an endo-loop is inserted into the interior of a human body together with the endoscope, a surgical portion is observed with the endoscope and is sutured with the endo-loop.

According to a conventional endo-loop used in a surgery which uses an endoscope, as shown in FIGS. 1A and 1B, a bar-shaped insert rod 11 which is thin, long, and hollow is inserted into the interior of a body together with an endoscope, a suturing thread 12′ used for suturing an internal organ, a blood vessel, or a nerve system is inserted into a body of the insert rod 11, and a grip integrally formed with the suturing thread 12′ extending through the interior of the insert rod 11 is provided at a rear end portion of the insert rod 11 and is detachably connected to the insert rod 11 by a cutting recess portion 14.

As shown FIG. 1A, a suturing thread loop 12 formed by making the suturing thread 12′ have a loop (ring) shape to suture a surgical portion is provided at the tip end portion of the insert rod 11. Further, as shown in FIG. 1B, the suturing thread 12′ itself extends by a predetermined distance outside the insert rod 11, and a suturing needle 15 is connected to the suturing thread 12′ to stitch an incised portion of a body.

FIG. 2 shows a representative embodiment of a suturing thread loop 12. As shown in FIG. 2, in order to perform a suturing surgery of an internal organ, a blood vessel, or a nerve system by using a conventional endo-loop 10, the internal organ, the blood vessel, or the nerve system is inserted into the suturing thread loop 12, and, if the grip portion 13 formed at the rear end portion of the insert rod 11 is bent with a hand, the grip 13 is separated from the insert rod 11 by the cutting recess portion 14, and, if the separated grip 13 is pulled to the rear side, the suturing thread 12′ integrally formed with the grip 13 is pulled to the rear side to tighten the suturing thread loop 12 formed at the tip end of the insert rod 11 and thus to suture the corresponding portion.

However, the conventional endo-loop 10 has a problem in that, after the suturing surgeries are completed with various suturing means by separating the grip 13 from the insert rod 11, the upper side of a portion in which the knot 12a is formed is cut away and the grip 13 of the insert rod 11 and the remaining suturing thread 12′ should be discarded. Therefore, the endo-loop 10 has a problem in that, in case a several kinds of suturing surgeries are required in one endoscopic surgery of one patient, considerably many endo-loops are consumed.

Namely, since, in one endoscopic surgery of one patent, two or three endo-loops 10 of various purposes are used in addition to the endo-loop of FIG. 1 and the different thickness and the different kind of the suturing thread 12′ used in suturing and stitching with the suturing thread loop 12 should be used according to the surgery portions and the surgery conditions, considerably many endo-loops 10 should be used and all the endo-loops used in the surgery should be discarded. Therefore, the endo-loop 10 has a problem in that, since the raw materials for surgeries are wasted and the amount of disused materials increases, it can cause environmental contamination.

Further, since, in an endoscopic surgery, in the state in which a plurality of endo-loops 10 shown in FIG. 1 are prepared, the endo-loops 10 themselves should be changed in every different suturing surgery. Complicated endoscopic surgeries become more troublesome and confusion may be caused between an operator, i.e. a doctor, and an assistant, i.e. a nurse due to the different kinds of endo-loops, and thus the endoscopic surgery cannot be smoothly performed.

In addition, even in a manufacturing aspect, since each endo-loop 10 shown in FIG. 1 should be packed in a separate container, the manufacturing cost cannot be reduced. Further, since the kind and the frequency of the suturing surgery using the corresponding endo-loop 10 is limited to one times, the feasibility and the functional characteristics lower. Furthermore, since most the endo-loops are imported, there is a need for securing product competitiveness and marketable aspect of the endo-loop 10 and a new technology development which can attribute to the localization of the product is required.

### DISCLOSURE OF INVENTION

Technical Problem

Therefore, the present invention has been made in view of the above-mentioned problems, and it is an object of the present invention to provide an endo-loop which can remarkably reduce the amount of disused surgery materials by minimizing the number of endo-loops used in an endoscopic surgery, can efficiently perform the endoscopic surgery by preventing problems due to exchanges of endo-loops in different surgeries and confusion in the surgery, can reduce the manufacturing cost of the endo-loop and maximize functionality and feasibility of the endo-loop by manufacturing almost all kinds of endo-loops in one packing unit, by extending outward a suturing thread to the rear side of an insert rod having a suturing means at the tip end of the insert rod, detachably engaging a suturing thread fixing member with the insert rod, and winding the suturing thread extending outward by a predetermined distance on a spool.

Technical Solution

According to an aspect of the present invention, there is provided an endo-loop for an endoscopic surgery, the endo-loop including: an insert rod inserted into a human body, the insert rod having a shape of a long, thin, and hollow bar; a suturing thread inserted through an interior of the insert rod; a suturing means provided at a tip end of the insert rod, the suturing means including at least one of the suturing thread, a suturing loop, a suturing needle connected to the suturing thread, and their combination; a suturing thread fixing member for fixing the suturing thread to the insert rod, the suturing thread extending backward along the insert rod, the suturing thread fixing member being detachably engaged with an open rear end portion of the inserted rod; and a spool on which the suturing thread extending outward through the open rear end portion of the insert rod is wound.

Advantageous Effects

According to the endo-loop for an endoscopic surgery of the present invention, since the suturing thread extends outward by a predetermined length to the rear side of the insert rod having the suturing means of the surgical portion at the tip end thereof, and the suturing thread fixing member fixing the suturing thread to the rear end portion of the insert rod is detachably engaged with the insert rod and the suturing thread extending outward by a predetermined length is wound on the spool, the number of the endo-loops used in one endoscopic surgery can be minimized and the waste of the raw materials and the amount of disused materials can be remarkably reduced.

Further, since almost all suturing surgeries which can be applied to one endoscopic surgery for one patient are to be performed with one endo-loop, problems due to the exchange of endo-loops at different surgical portions and confusion in the endoscopic surgery are prevented and thus the endoscopic surgery is performed more efficiently.

Furthermore, since almost all kinds of endo-loops which can be applied to endoscopic surgeries can be manufactured in a packing unit, the manufacturing cost of the endo-loop lowers and the functionality and the feasibility of the product maximizes.

Therefore, the present invention attributes to the localization of the endo-loop products which have almost been imported.

While this invention has been described in connection with what is presently considered to be the most practical and preferred embodiment, it is to be understood that the invention is not limited to the disclosed embodiment and the drawings, but, on the contrary, it is intended to cover various modifications and variations within the spirit and scope of the appended claims.

### BRIEF DESCRIPTION OF THE DRAWINGS

The foregoing and other objects, features and advantages of the present invention will become more apparent from the following detailed description when taken in conjunction with the accompanying drawings in which:

FIGS. 1A and 1B are perspective views showing conventional endo-loops;

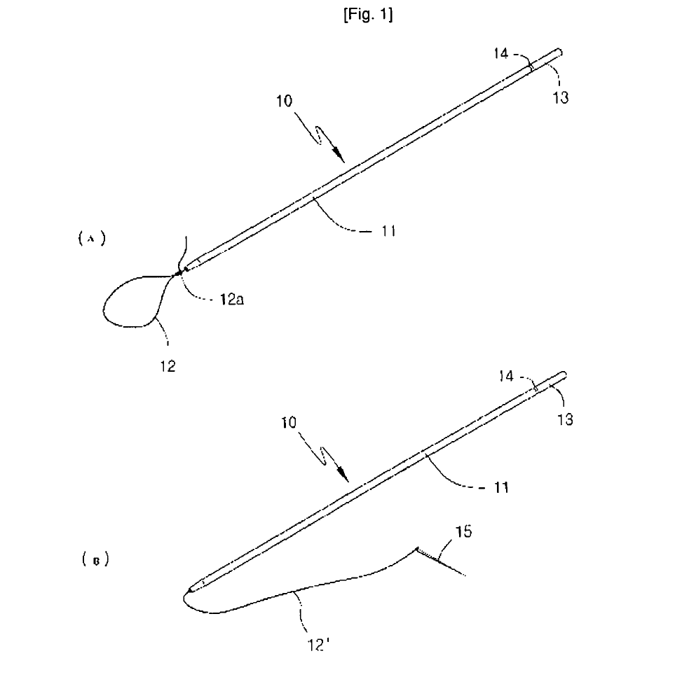

FIG. 2 is a partially exploded side cross-sectional view for showing an application state of a conventional endo-loop;

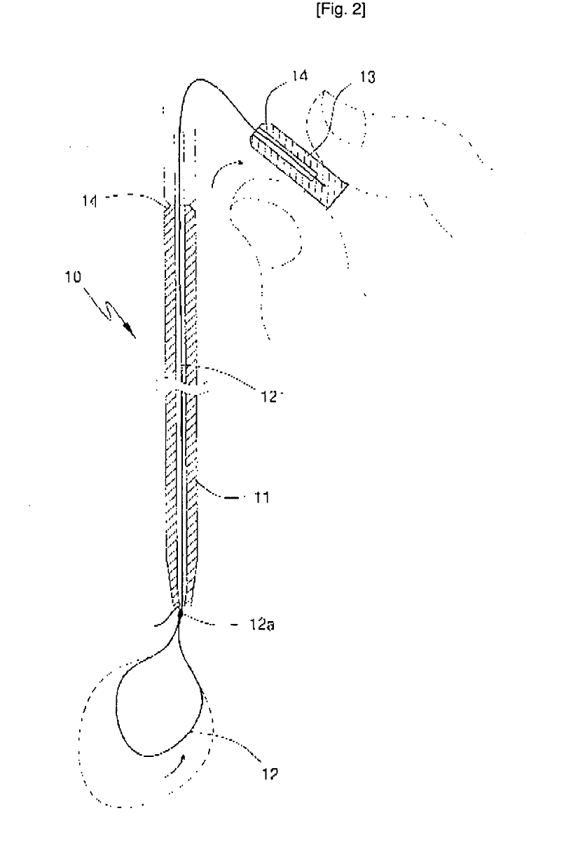

FIGS. 3A to 3C are perspective views according to the present invention;

FIGS. 4A and 4B are partially exploded side cross-sectional views for showing combination states of suturing thread fixing members of endo-loops according to the present invention; and

FIG. 5 is a perspective view for showing a surgery set using an endo-loop according to the present invention.

### MODE FOR THE INVENTION

Hereinafter, the present invention will be described in detail with reference to accompanying drawings.

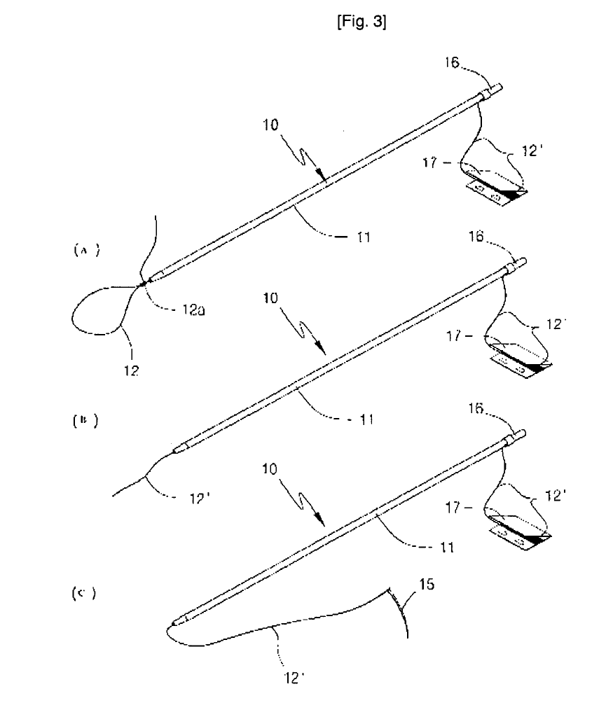

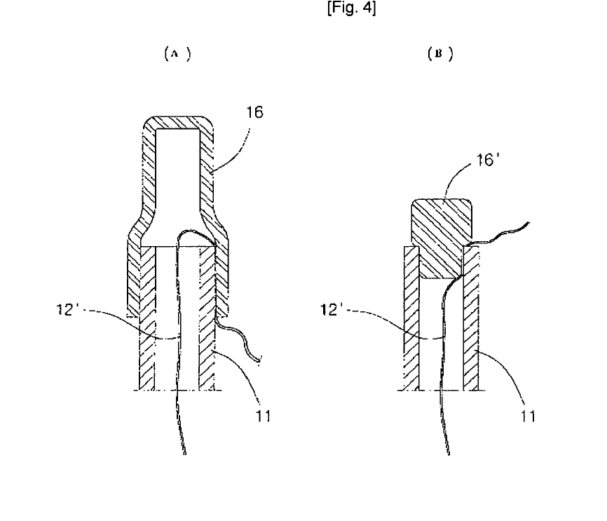

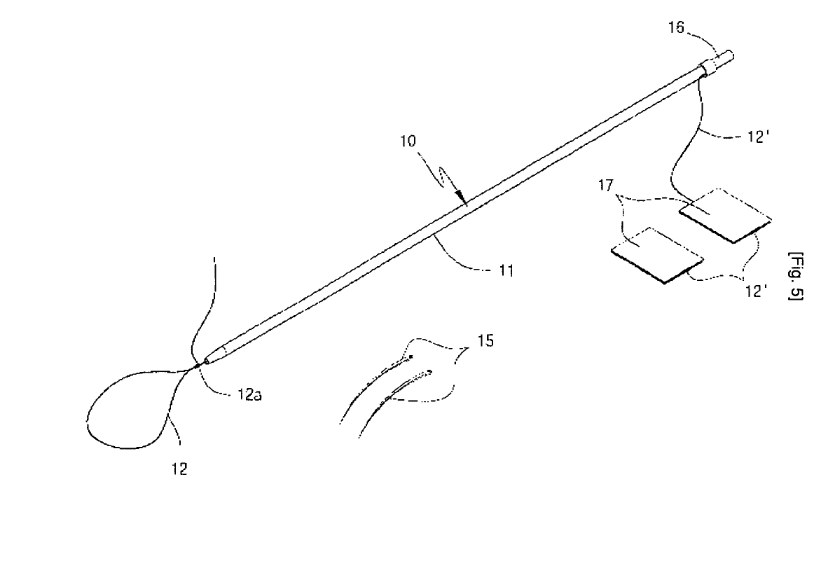

FIGS. 3A to 3C are perspective views according to the present invention, and FIGS. 4A and 4B are partially exploded side cross-sectional views for showing combination states of suturing thread fixing members of endo-loops according to the present invention, and FIG. 5 is a perspective view for showing a surgery set using an endo-loop according to the present invention. The reference numeral 12a represents a knot for a suturing thread loop.

According to an endo-loop for an endoscopic surgery, as shown in FIG. 3, a bar-shaped insert rod 11 which is thin, long, and hollow is formed to be inserted into the interior of a body together with an endoscope, and a suturing thread 12′ is inserted into the interior of the insert rod 11 along the body of the insert rod 11, and various suturing means cooperated with the suturing thread 12′ are provided at the tip end of the insert rod 11, as described in the background art.

Namely, the suturing means include a suturing loop 12 for suturing a surgical portion by making the suturing thread 12′ have a loop (ring) shape as shown in FIG. 3A, an extending suturing thread 12′ for binding both sides of an incising portion before incising an internal organ, a blood vessel, or a nerve system by extending outward shown in FIG. 3B, and a suturing needle 15 connected to the suturing thread 12′ to stitch an incised portion of a body as shown in FIG. 3C.

FIGS. 3A to 3C show three suturing means widely applied to an endo-loop representatively, and other suturing means can be formed. As will be explained hereinafter, according to the endo-loop 10 of the present invention, after performing a suturing surgery of a surgical portion by using the suturing means formed at the tip end of the insert rod 11, the suturing thread 12′ is withdrawn by a predetermined distance through the insert rod 11. Therefore, since the suturing means can be directly made to be used irrespective of the number of suturing surgeries, the kinds of the suturing means formed at the tip end of the insert rod 11 is almost irrelevant.

The insert rod 11, the main component of the endo-loop of the present invention, has an open rear end portion. At the open rear end portion of the insert rod, detachably combined is a suturing thread fixing member 16 for fixing and adhering to the insert rod 11 the suturing thread 12′ extending along the interior of the insert rod 11. The suturing thread 12′ extending outward by a predetermined distance through the open rear end portion of the insert rod 11 is kept wound on a spool 17.

It is preferable that the length of the suturing thread 12′ extending outward through the open rear end portion of the insert rod 11 is limited within 10 to 50 cm so that several suturing surgeries can be performed by using one endo-loop 10 during one endoscopic surgery of one patient. If the suturing thread 12′ extends by a length of under 10 cm, the number of suturing surgeries using one endo-loop is limited to twice or three times, which is not preferable. The longer the extending length of the suturing thread 12′ is, the more the number of suturing surgeries using one endo-loop.

However, the extending length of the suturing thread 12′ is limited to under 50 cm, considering the waste of the suturing thread 12′ and the number of general suturing surgeries which can be applied to one endoscopic surgery.

The spool 17 is provided to prevent the suturing thread 12′ from being scattered around the surgery place during a suturing surgery using the endo-loop 10 according to the present invention. The suturing thread 12′ is unraveled from the spool 17 by a length needed for one surgery. The spool 17 can have an arbitrary shape except for the shape shown in FIG. 3, only if the suturing thread 12′ can be wound around it. Further, the material of the spool 17 does not matter only if the suturing thread 12′ is not damaged by the spool 17 or it does hinder the surgery.

The suturing thread 12′ extending outward through the open rear end portion of the insert rod 11 is adhered to and fixed to the insert rod 11 by using the suturing thread fixing member 16 and 16′ As shown in FIG. 4A, the lid-shaped suturing thread fixing member 16 is combined outside the rear end portion of the insert rod 11 to adhere and fix the suturing thread 12′ to the outer surface of the insert rod 11. As shown in FIG. 4B, the packing-shaped suturing thread fixing member 16 is inserted into the rear end portion of the insert rod 11 to adhere and fix the suturing thread 12′ to the inner surface of the insert rod 11.

The suturing thread fixing member 16 and 16′ can be made of various materials such as paper, rubber, plastic, and cork, but an elastic rubber material is preferably used to prevent damage of the suturing thread 12′ adhered to the insert rod 11. However, in some cases, by forming a cut-off recess at the rear end portion of the insert rod 11, the suturing thread 12′ can be inserted into and fixed to the cut-off recess.

Hereinafter, operation of the endo-loop according to the present invention will be explained in detail with reference to the accompanying drawings. In the explanation of the operation of the present invention, the first suturing surgery using the endo-loop in which the suturing thread loop 12 is used as the suturing means is described as a representative embodiment.

After the endo-loop 10 according to the present invention including the suturing loop 12 as the suturing means is inserted into the corresponding surgical portion together with an endoscope, the incising portion of an internal organ, a blood vessel, or a nerve system is inserted through the suturing thread loop 12. Then, the suturing thread fixing member 16 and 16′ engaged with the rear end portion of the insert rod 11 is separated from the insert rod 11. Then, if the suturing thread 12′ is pulled rearward, the suturing thread loop 12 formed at the tip end of the insert rod 11 is tightened and the corresponding incising portion is sutured. Thereafter, if the knot 12a forming the suturing thread loop 12 is cut by scissors, the suturing surgery using the endo-loop 10 is completed.

After the first suturing surgery is completed, the suturing thread 12′ is unraveled from the spool 17 by the length to be used at the second suturing surgery and the unraveled suturing thread 12′ is pushed into the inserted rod 11, the suturing thread 12′ is exposed through the tip end portion of the insert rod 11 by a predetermined length. In this manner, the suturing thread 12′ is withdrawn from the insert rod 11 by a required length. Then, in the state in which the suturing thread 12′ is fixed to the insert rod 11 by engaging the suturing thread fixing member 16 and 16′ with the insert rod 11, the suturing thread 12′ is used as it is, the suturing loop 12 is formed by the knot 12a, or the suturing thread loop 12 is inserted into the suturing needle 15 to form the suturing means for the second surgery.

As above-described, after the suturing means for the second surgery is formed at the tip end of the insert rod 11 of the endo-loop and one endoscopic surgery for one patient is completed, the endo-loop 10 of the present invention should be disused.

According to the present invention, only one insert rod 11 and the suturing thread 12′ left after the surgery are disused, and the suturing thread fixing member 16, and 16′ which have not made contact with the surgical portion and the spool can be reused. Therefore, the materials disused in the surgery can be minimized, and thus waste of the raw materials and the environmental contamination due to the disused materials can be effectively prevented.

Further, on the basis of one endo-loop 10 according to the present invention as shown in FIG. 5, if the suturing thread 12′ provided in the endo-loop 10, the spool 17 on which the suturing thread 12′ is wound, and the suturing needle 15 is used as a set, almost all kinds of endo-loops 10 can be directly made and used with the set. Then, the endo-loops 10 need not be exchanged at the respective surgery in the state a plurality of suturing threads 12′ are prepared at different kinds. Further, Confusion between the operator, i.e. the doctor, and the assistant, i.e. a nurse, can be minimized and thus the endoscopic surgery can be performed efficiently.

In addition, even in the manufacturing aspect of the endo-loop 10, the set shown in FIG. 5 can become a packing unit and thus the mass-production is enabled. Therefore, the productivity of the endo-loop 10 improves and the manufacturing cost lowers. Further, since the kinds and the number of the suturing surgery using the endo-loop set shown in FIG. 5 covers the entire endoscopic surgery with respect to one patient and one surgery, the feasibility and the functionality of the endo-loop 10 remarkably improves. Therefore, by securing the product competitiveness and marketable aspect, the present invention attributes to the localization of the endo-loops 10 almost all of which have been imported.

1. An endo-loop for an endoscopic surgery, the endo-loop comprising:
an insert rod inserted into a human body, the insert rod having a shape of a long, thin, and hollow bar;
a suturing thread inserted through an interior of the insert rod;
a suturing means provided at a tip end of the insert rod, the suturing means including at least one of the suturing thread, a suturing loop, a suturing needle connected to the suturing thread, and their combination;
a suturing thread fixing member for fixing the suturing thread to the insert rod, the suturing thread extending backward along the insert rod, the suturing thread fixing member being detachably engaged with an open rear end portion of the inserted rod; and
a spool on which the suturing thread extending outward through the open rear end portion of the insert rod is wound.
